(19) United States
(12) Patent Application Publication
(10) Pub. No.: US 2016/0015700 A1
(43) Pub. Date: Jan. 21, 2016

(54) 4-CYANO-N-(2-(4,4-DIMETHYLCYCLOHEX-1-EN-1-YL)-6-(2,2,6,6-TETRAMETHYLTETRAHYDRO-2H-PYRAN-4-YL)PYRIDIN-3-YL)-1H-IMIDAZOLE-2-CARBOXAMIDE FOR THE TREATMENT OF HODGKIN'S LYMPHOMA

(71) Applicants: Carla DE BOER, Leiden (NL); Yusri A. ELSAYED, (US); Imran KHAN, (US); Carl L. MANTHEY, Chester Springs, PA (US); Linda SYNDER, Pottstown, PA (US); JANSSEN PHARMACEUTICA NV, Beerse (BE)

(72) Inventors: Carla DE BOER, Leiden (NL); Yusri A. ELSAYED, Spring House, PA (US); Imran KHAN, Raritan, NJ (US); Carl L. MANTHEY, Chester Springs, PA (US); Linda SNYDER, Pottstown, PA (US)

(21) Appl. No.: 14/772,027

(22) PCT Filed: Mar. 13, 2014
(86) PCT No.: PCT/US14/25297

§ 371 Date: Sep. 1, 2015

Related U.S. Application Data
(60) Provisional application No. 61/792,259, filed on Mar. 15, 2013.

(51) Int. Cl. A61K 31/4439; A61K 31/573; A61K 31/7048; A61K 31/704; A61K 31/655; A61K 31/475; A61K 38/14; A61K 45/06; A61K 31/675

(52) U.S. Cl. CPC A61K 31/4439; A61K 45/06; A61K 31/573; A61K 31/7048; A61K 31/675; A61K 31/655; A61K 31/475; A61K 38/14; A61K 31/704

(57) ABSTRACT

The present invention is directed to methods for the treatment of Hodgkin's lymphoma comprising administering to a patient in need thereof, a therapeutically effective amount of 4-cyano-N-(2-(4,4-dimethylcyclohex-1-en-1-yl)-6 -(2,2,6,6-tetramethyltetrahydro-2H-pyran-4-yl)pyridin-3-yl)-1H-imidazole-2-carboxamide as mono-therapy or as combination or co-therapy with one or more chemotherapeutic agent or chemotherapy regimens.

### CROSS REFERENCE TO RELATED APPLICATIONS

This application claims the benefit of U. S. Provisional Application 61/792,259, filed on Mar. 15, 2013, which is incorporated by reference herein in its entirety.

### FIELD OF THE INVENTION

The present invention is directed to methods for the treatment of Hodgkin's Lymphoma comprising administering to a patient in need thereof, a therapeutically effective amount of 4-cyano-N-(2-(4,4-dimethylcyclohex-1-en-1-yl)-6-(2,2,6,6-tetramethyltetrahydro-2H-pyran-4-yl)pyridin-3-yl)-1H-imidazole-2-carboxamide, as mono-therapy or co-therapy with one or more chemotherapeutic agent or chemotherapy regimen.

### BACKGROUND OF THE INVENTION

Hodgkin's lymphoma, also known as Hodgkin lymphoma and previously known as Hodgkin's disease, is a type of lymphoma, which is a cancer originating from white blood cells called lymphocytes. Hodgkin's lymphoma is characterized by the orderly spread of disease from one lymph nodes group to another and by the development of systemic symptoms with advanced disease. When Hodgkins cells are examined microscopically, multinucleated Reed-Sternberg cells (RS cells) are the characteristic histopathologic finding. The disease occurrence shows two peaks: the first in young adulthood (age 15-35) and the second in those over 55 years old.

Hodgkin lymphoma is classified in 2 subtypes: classical Hodgkin lymphoma (cHL, 95%) and lymphocyte predominant Hodgkin lymphoma (LPHL, 5%). Classical Hodgkin's lymphoma (excluding nodular lymphocyte predominant Hodgkin's lymphoma) can be subclassified into 4 pathologic subtypes based upon Reed-Sternberg cell morphology and the composition of the reactive cell infiltrate seen in the lymph node biopsy specimen (the cell composition around the Reed-Sternberg cell(s)).

(a) Nodular sclerosing HL: the most common subtype, composed of large tumor nodules showing scattered lacunar classical RS cells set in a background of reactive lymphocytes, eosinophils and plasma cells with varying degrees of collagen fibrosis/sclerosis.

(b) Mixed-cellularity subtype: a common subtype composed of numerous classic RS cells admixed with numerous inflammatory cells including lymphocytes, histiocytes, eosinophils, and plasma cells without sclerosis. This type is most often associated with EBV infection and may be confused with the early, so-called ‘cellular’ phase of nodular sclerosing CHL.

(c) Lymphocyte—rich or Lymphocytic predominance: a rare subtype, shows many features which may cause diagnostic confusion with nodular lymphocyte predominant B-cell Non-Hodgkin's Lymphoma (B-NHL). This form also has the most favorable prognosis.

(d) Lymphocyte depleted: a rare subtype, composed of large numbers of often pleomorphic RS cells with only few reactive lymphocytes which may easily be confused with diffuse large cell lymphoma. Many cases previously classified within this category would now be reclassified under anaplastic large cell lymphoma.

(e) Unspecified subtype.

Hodgkin's lymphoma may be treated with radiation therapy, chemotherapy or hematopoietic stem cell transplantation, with the choice of treatment depending on the age and sex of the patient and the stage, bulk, and histological subtype of the disease.

Hodgkin lymphoma is curable with modern treatment strategies, although approximately 20% will die after relapse. The incidence of classical Hodgkin's Lymphoma (cHL) with age follows a bimodal distribution peaking at 15 to 44 years of age (3.35 per 100,000 persons, 95% confidence interval [CI]: 2.56; 3.05) and 65 to 74 years of age (2.80 per 100,000 persons, 95% CI: 2.56; 3.05). The median age at diagnosis is 38 years of age. Over 12% (12.3%) of cHL cases occur in patients under 20 years of age.

The overall 5-year relative survival for 2001-2007 from 17 SEER geographic areas was 83.9%. Since many patients are young, they often live 40 years or more after treatment. However, few studies follow patients as long as 25 years, and those studies are of older treatments with more life-threatening adverse effects. There is insufficient data available about the long-term outcomes of newer, less-toxic regimens and ones which limit radiation exposure. Radiation treatments, and some chemotherapy drugs, pose a risk of causing potentially fatal secondary cancers, heart disease, and lung disease 40 or more years later. Modern treatments greatly minimize the chances of these late effects.

The staging is the same for both Hodgkin's as well as non-Hodgkin's lymphomas. On the basis of this staging, the patient is classified according to a staging classification (the Ann Arbor staging classification scheme is a common one):
(a) Stage I is involvement of a single lymph node region (I) (mostly the cervical region) or single extralymphatic site (Ie);
(b) Stage II is involvement of two or more lymph node regions on the same side of the diaphragm (II) or of one lymph node region and a contiguous extralymphatic site (IIe);
(c) Stage III is involvement of lymph node regions on both sides of the diaphragm, which may include the spleen (IIIs) and/or limited contiguous extralymphatic organ or site (IIIe, IIIes);
(d) Stage IV is disseminated involvement of one or more extralymphatic organs.

Patients with early stage disease (IA or IIA) are effectively treated with radiation therapy or chemotherapy. The choice of treatment depends on the age, sex, bulk and the histological subtype of the disease. Patients with later disease (III, IVA, or IVB) are treated with combination chemotherapy alone. Patients of any stage with a large mass in the chest are usually treated with combined chemotherapy and radiation therapy.

Chemotherapy regimen used in the treatment of Hodgkin's Lymphona include, ABVD (doxorubicin, bleomycin, vinblastine, dacarbazine), BEACOPP (doxorubicin, bleomycin, vincristine, cyclophosphamide, procarbazine, etoposide, prednisone) and Standford V (doxorubicin, bleomycin, vinblastine, vincristine, mechlorethamine, etoposide, prednisone). Approved monotherapy and combination therapy for the treatment of Hodgkin's lymphoma are available from government and medical source, including for example, the National Cancer Institute at the NIH.

There remains a need to provide an effective treatment for Hodgkin's Lymphoma.

### SUMMARY OF THE INVENTION

The present invention is directed to methods for the treatment of Hodgkin's Lymphoma, comprising administering to a patient in need thereof, a therapeutically effective amount of a compound of formula (I)

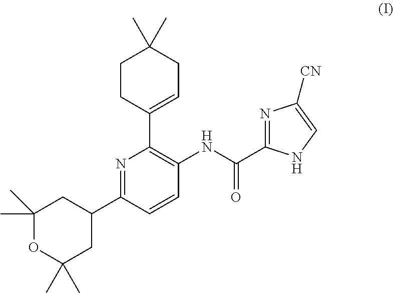 [SMILES: CC1(C)CC=C(c2nc(C3CC(C)(C)OC(C)(C)C3)ccc2NC(=O)c2nc(C#N)c[nH]2)CC1]

also known as 4-cyano-N-(2-(4,4-dimethylcyclohex-1-en-1-yl)-6-(2,2,6,6-tetramethyltetrahydro-2H-pyran-4-yl)pyridin-3-yl)-1H-imidazole-2-carboxamide (as disclosed in Illig, C., et al., in US Patent Publication US2009/0105296 A1, published Apr. 23, 2009); or a solvate, hydrate, tautomer, or pharmaceutically acceptable salt thereof.

The compound of formula (I) is a protein tyrosine kinase inhibitors, more particularly as an inhibitor of c-fms kinase. As disclosed in Illig, C., et al., US Patent Publication US2009/0105296 A1, the c-fms kinase inhibitor of formula (I) is useful for the treatment of diseases including, but not limited to: osteoporosis, Paget's disease, rheumatoid arthritis, other forms of inflammatory arthritis, osteoarthritis, prosthesis failure, osteolytic sarcoma, myeloma, tumor metastasis to bone, ovarian cancer, uterine cancer, breast cancer, prostate cancer, lung cancer, colon cancer, stomach cancer, hairy cell leukemia; metastasis from ovarian cancer, uterine cancer, breast cancer, prostate cancer, lung cancer, colon cancer, stomach cancer, or hairy cell leukemia; glomerulonephritis, inflammatory bowel disease, sarcoidosis, congestive obstructive pulmonary disease, idiopathic pulmonary fibrosis, asthma, pancreatitis, HIV infection, psoriasis, diabetes, tumor related angiogenesis, age-related macular degeneration, diabetic retinopathy, restenosis, schizophrenia, Alzheimer's dementia; pain, skeletal pain caused by tumor metastasis or osteoarthritis, visceral pain, inflammatory pain, neurogenic pain; an autoimmune disease, systemic lupus erythematosus, rheumatoid arthritis, other forms of inflammatory arthritis, psoriasis, Sjogren's syndrome, multiple sclerosis and uveitis.

The present invention is further directed to a method for the treatment of Hodgkin's Lymphoma comprising administering to a patient in need thereof, combination or co-therapy with a therapeutically effective amount of a compound of formula (I), or a solvate, hydrate, tautomer, or pharmaceutically acceptable salt thereof; and at least one chemotherapeutic agent or chemotherapy regimen, as herein defined.

In an embodiment, the chemotherapeutic agents are each independently selected from the group consisting of doxorubicin, bleomycin, vinblastine, vincristine, dacarbazine, mechlorethamine, cyclophosphamide, procarbazine, etoposide and prednisone. In another embodiment, the chemotherapy regimen is selected from the group consisting of ABVD, BEACOPP and Standford V.

The present invention is further directed to the use of the compound of formula (I) in the preparation of a medicament for treating Hodgkin's Lymphoma, in a patient in need thereof. The present invention is further directed to the compound of formula (I) for use in a method for the treatment of Hodgkin's Lymphoma, in a subject in need thereof. In another embodiment, the present invention is directed to a composition comprising the compound of formula (I)for the treatment of Hodgkin's Lymphoma.

### DETAILED DESCRIPTION OF THE INVENTION

The present invention is directed to a method for the treatment of Hodgkin's Lymphoma, comprising administering to a patient in need thereof, a therapeutically effective amount of the compound of formula (I). In an embodiment, the present invention is directed to a method for the treatment of relapsed or refractory Hodgkin's Lymphoma, comprising administering to a patient in need thereof, a therapeutically effective amount of the compound of formula (I).

The present invention is further directed to a method for the treatment of Hodgkin's Lymphoma comprising administering to a patient in need thereof, combination or co-therapy comprising the compound of formula (I) and one or more chemotherapeutic agent or a chemotherapy regimen; preferably in combination with or as co-therapy with a chemotherapy regimen. In an embodiment, the chemotherapy regimen is selected from the group consisting of ABVD, BEACOPP and Standford V.

In certain embodiments of the present invention, the compound of formula (I) may be administered in combination with one or more chemotherapeutic agents, as herein described, preferably in combination with one to three chemotherapeutic agents. In another embodiment, the present invention is directed to combination or co-therapy comprising a compound of formula (I) and doxorubicin. In another embodiment, the present invention is directed to combination or co-therapy comprising a compound of formula (I), doxorubicin, bleomycin and vinblastine.

As used herein, unless otherwise noted, the term “chemotherapeutic agents” shall mean any pharmaceutical agent which has shown therapeutic efficacy, alone or in combination with other pharmaceutical agents, in the treatment of Lymphoma, preferably, in the treatment of Hodgkin's lymphoma. Suitable examples include, but are not limited to, doxorubicin, bleomycin, vinblastine, vincristine, dacarbazine, mechlorethamine, cyclophosphamide, procarbazine, etoposide and prednisone.

As used herein, the terms “chemotherapy regime” and “chemoregime” shall mean any regimen comprising two or more chemotherapeutic agents which are administered as combination or co-therapy for the treatment of Hodgkin's Lymphoma. Suitably examples include, but are not limited to

(a) ABVD, which comprises administration of doxorubicin, bleomycin, vinblastine and dacarbazine;

(b) BEACOPP, which comprises administration of doxorubicin, bleomycin, vincristine, cyclophosphamide, procarbazine, etoposide and prednisone; and

(c) Standford V, which comprises administration of doxorubicin, bleomycin, vinblastine, vincristine, mechlorethamine, etoposide and prednisone.

Preferably, the chemotherapy regime is selected from the group consisting of ABVD, BEACOPP and Standford V.

In an embodiment, the present invention is directed to methods for the treatment of Hodgkin's Lymphoma, wherein the compound of formula (I) is administered at a dosage, preferably at a daily dosage, amount in the range of from about 10 mg to about 1000 mg, or any amount or range therein, preferably from about 500 mg to about 600 mg, or any amount or range therein. In another embodiment, the present invention is directed to methods for the treatment of Hodgkin's Lymphoma, wherein the compound of formula (I) is administered at a dosage, preferably at a daily dosage, amount in the range of from about 10 mg to about 1000 mg, or any amount or range therein, preferably, in an amount selected from the group consisting of 10 mg, 25 mg, 50 mg, 100 mg, 200 mg, 300 mg, 400 mg, 500 mg, 600 mg, 750 mg and 1000 mg.

As used herein, unless otherwise noted, the terms “subject” and “patient” refer to an animal, preferably a mammal, most preferably a human, who has been the object of treatment, observation or experiment. Preferably, the subject or patient has experienced and/or exhibited at least one symptom of the disease or disorder to be treated and/or prevented.

As used herein, unless otherwise noted, the terms “treating”, “treatment” and the like, shall include the management and care of a subject or patient (preferably mammal, more preferably human) for the purpose of combating a disease, condition, or disorder and includes the administration of a compound of the present invention to prevent the onset of the symptoms or complications, alleviate the symptoms or complications, or eliminate the disease, condition, or disorder.

As used herein, unless otherwise noted, the term “prevention” shall include (a) reduction in the frequency of one or more symptoms; (b) reduction in the severity of one or more symptoms; (c) the delay or avoidance of the development of additional symptoms; and/or (d) delay or avoidance of the development of the disorder or condition.

One skilled in the art will recognize that wherein the present invention is directed to methods of prevention, a subject in need of thereof (i.e. a subject in need of prevention) shall include any subject or patient (preferably a mammal, more preferably a human) who has experienced or exhibited at least one symptom of the disorder, disease or condition to be prevented. Further, a subject in need thereof may additionally be a subject (preferably a mammal, more preferably a human) who has not exhibited any symptoms of the disorder, disease or condition to be prevented, but who has been deemed by a physician, clinician or other medical profession to be at risk of developing said disorder, disease or condition. For example, the subject may be deemed at risk of developing a disorder, disease or condition (and therefore in need of prevention or preventive treatment) as a consequence of the subject's medical history, including, but not limited to, family history, pre-disposition, co-existing (comorbid) disorders or conditions, genetic testing, and the like.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

Wherein the present invention is directed to therapy with a combination of agents, “therapeutically effective amount” shall mean that amount of the combination of agents taken together so that the combined effect elicits the desired biological or medicinal response. For example, the therapeutically effective amount of combination therapy comprising the compound of formula (I) and a chemotherapeutic agent would be the amount of the compound of formula (I) and the amount of the chemotherapeutic agent that when taken together or sequentially have a combined effect that is therapeutically effective, more preferably where the combined effect is synergistic. Further, it is recognized by one skilled in the art that in the case of combination therapy with a therapeutically effect amount, the amount of each component of the combination individually may or may not be therapeutically effective.

Wherein the present invention is directed to the administration of a combination, the compounds may be co-administered simultaneously, sequentially, separately or in a single pharmaceutical composition. Where the compounds are administered separately, the number of dosages of each compound given per day, may not necessarily be the same, e.g. where one compound may have a greater duration of activity, and will therefore, be administered less frequently. Further, the compounds may be administered via the same or different routes of administration, and at the same or different times during the course of the therapy, concurrently in divided or single combination forms. The instant invention is therefore understood as embracing all regimens of simultaneous or alternating treatment and the term “administering” is to be interpreted accordingly.

As used herein, the terms “co-therapy”, “combination therapy”, “adjunctive treatment”, “adjunctive therapy” and “combined treatment” shall mean treatment of a patient in need thereof by administering the compound of formula (I) in combination with one or more chemotherapeutic agent(s), wherein the compound of formula (I) and the chemotherapeutic agent(s) are administered by any suitable means, simultaneously, sequentially, separately or in a single pharmaceutical formulation. Where the compound of formula (I) and the chemotherapeutic agent(s) are administered in separate dosage forms, the number of dosages administered per day for each compound may be the same or different. The compound of formula (I) and the chemotherapeutic agent(s) may be administered via the same or different routes of administration. Examples of suitable methods of administration include, but are not limited to, oral, intravenous (iv), intranasal (in) intramuscular (im), subcutaneous (sc), transdermal, and rectal. Compounds may also be administered directly to the nervous system including, but not limited to, intracerebral, intraventricular, intracerebroventricular, intrathecal, intracisternal, intraspinal and/or peri-spinal routes of administration by delivery via intracranial or intravertebral needles and/or catheters with or without pump devices. The compound of formula (I) and the chemotherapeutic agent(s) may be administered according to simultaneous or alternating regimens, at the same or different times during the course of the therapy, concurrently in divided or single forms.

Optimal dosages to be administered may be readily determined by those skilled in the art, and will vary with the particular compound or compounds used, the mode of administration, the strength of the preparation and the advancement of the disease condition. In addition, factors associated with the particular patient being treated, including patient's sex, age, weight, diet, time of administration and concomitant diseases, will result in the need to adjust dosages.

One skilled in the art will recognize that, both in vivo and in vitro trials using suitable, known and generally accepted cell and/or animal models are predictive of the ability of a test compound to treat or prevent a given disorder.

One skilled in the art will further recognize that human clinical trails including first-in-human, dose ranging and efficacy trials, in healthy patients and/or those suffering from a given disorder, may be completed according to methods well known in the clinical and medical arts.

As used herein, the term “composition” is intended to encompass a product comprising the specified ingredients in the specified amounts, as well as any product which results, directly or indirectly, from combinations of the specified ingredients in the specified amounts.

The present invention further comprises pharmaceutical compositions for the treatment of Hodgkin's lymphoma containing the compound of formula (I), optionally in combination with one or more chemotherapeutic agents, with a pharmaceutically acceptable carrier. Pharmaceutical compositions containing one or more of the compounds of the invention described herein as the active ingredient can be prepared by intimately mixing the compound or compounds with a pharmaceutical carrier according to conventional pharmaceutical compounding techniques. The carrier may take a wide variety of forms depending upon the desired route of administration (e.g., oral, parenteral). Thus for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Solid oral preparations may also be coated with substances such as sugars or be enteric-coated so as to modulate major site of absorption. For parenteral administration, the carrier will usually consist of sterile water and other ingredients may be added to increase solubility or preservation. Injectable suspensions or solutions may also be prepared utilizing aqueous carriers along with appropriate additives.

To prepare the pharmaceutical compositions of this invention, the compound of formula (I), and optionally, at least one chemotherapeutic agent, as the active ingredient(s) are intimately admixed with a pharmaceutical carrier according to conventional pharmaceutical compounding techniques, which carrier may take a wide variety of forms depending of the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, caplets, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, through other ingredients, for example, for purposes such as aiding solubility or for preservation, may be included. Injectable suspensions may also be prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein will contain, per unit dosage unit, e.g., tablet, capsule, powder, injection, suppository, teaspoonful, and the like, of from about 10 mg to about 1000 mg or any amount or range therein, and may be given at a dosage, preferably at a daily dosage, of from about 0.1 mg/kg to about 15.0 mg/kg, or any amount or range therein, preferably from about 0.5 mg/kg to about 10.0 mg/kg, or any amount or range therein, preferably from about 1.0 mg/kg to about 10.0 mg/kg, or any amount or range therein, of each active ingredient. The dosages, however, may be varied depending upon the requirement of the patients, the severity of the condition being treated and the compound being employed. The use of either daily administration or post-periodic dosing may be employed.

Preferably these compositions are in unit dosage forms from such as tablets, pills, capsules, powders, granules, sterile parenteral solutions or suspensions, metered aerosol or liquid sprays, drops, ampoules, autoinjector devices or suppositories; for oral parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective dosage forms such as tablets, pills and capsules. This solid preformulation composition is then subdivided into unit dosage forms of the type described above containing from about 10.0 mg to about 1,000 mg, or any amount or range therein, of each active ingredient. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

The liquid forms in which the novel compositions of the present invention may be incorporated for administration orally or by injection include, aqueous solutions, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin.

The method of treating Hodgkin's lymphoma described in the present invention may also be carried out using a pharmaceutical composition comprising any of the compounds as defined herein and a pharmaceutically acceptable carrier. The pharmaceutical composition may contain between about 10.0 mg and about 1000 mg of the compound, or any amount or range therein; preferably from about 50 mg to about 600 mg of the compound, or any amount or range therein, of each active ingredient, and may be constituted into any form suitable for the mode of administration selected. Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixers, emulsions, and suspensions. Forms useful for parenteral administration include sterile solutions, emulsions and suspensions.

Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal skin patches well known to those of ordinary skill in that art. To be administered in the form of a transdermal delivery system, the dosage administration will, of course, be continuous rather than intermittent throughout the dosage regimen.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms in suitably flavored suspending or dispersing agents such as the synthetic and natural gums, for example, tragacanth, acacia, methyl-cellulose and the like. For parenteral administration, sterile suspensions and solutions are desired. Isotonic preparations which generally contain suitable preservatives are employed when intravenous administration is desired.

To prepare a pharmaceutical composition of the present invention, the compound of formula (I), optionally in combination with at least one chemotherapeutic agent, as the active ingredient is intimately admixed with a pharmaceutical carrier according to conventional pharmaceutical compounding techniques, which carrier may take a wide variety of forms depending of the form of preparation desired for administration (e.g. oral or parenteral). Suitable pharmaceutically acceptable carriers are well known in the art. Descriptions of some of these pharmaceutically acceptable carriers may be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

The following Examples are set forth to aid in the understanding of the invention, and are not intended and should not be construed to limit in any way the invention set forth in the claims which follow thereafter.

### EXAMPLE 1

### Case Study

A classical Hodgkin's Lymphoma patient, diagnosed with Stage II in Hodgkin Lymphoma in January 2006, and previously treated with radiotherapy and three different regimens of chemotherapy, including an autologous stem cell transplantation was treated with 150 mg of the compound of formula (I), administered orally 1 time a day. In addition to the compound of formula (I), the patient was taking the following medications and dosages: (a) DEKRISTOL (cholecalciferol), 20000 I.U. QOD for vitamin deficiency, (b) methylphenidate hydrochloride, 20 mg DAILY for fatigue, (c) ibuprofen, 600 mg PRN for back pain, and (d) VALORON N (tilidine) 50 IN 20 DROPS PRN for back pain.

Response to treatment was evaluated by tumor assessments using CT/MRI scan and PET scan, resulting in an individual response for each technique. In addition, an overall response evaluation was completed, taking into account both scan results. Response evaluations were completed according to the criteria as set forth in CHESON, B. D., et al., J. Clin. Oncology, 2007, pp579-586, Vol. 25(5).

After 121 days of treatment with the compound of formula (I), the patient was evaluated and found to have complete response to treatment. As of 13 Mar. 2013, the patient was still in complete response as defined in CHESON, B. D., et al., J. Clin. Oncology, 2007, pp 579-586, Vol. 25(5) as disappearance of all detectable clinical evidence of disease and disease related symptoms if present before therapy.

### EXAMPLE 2

### Prophetic Example

### Clinical Trial Protocol: Treatment of Relapse or Refractory Hodgkin's Lymphoma

The ability of the compound of formula (I) to treat Hodgkin's lymphoma is evaluated via a suitably designed clinical study, as briefly summarized below. A copy of the complete clinical trial protocol is attached herewith.

This is an Open-label, Multicenter, Phase ½ Study of the compound of formula (I), an FMS Inhibitor, in Subjects with Relapsed or Refractory Hodgkin Lymphoma.

### Primary Objectives

Phase 1: To establish the recommended Phase 2 dose for the compound of formula (I).

Phase 2: To determine the overall response rate (complete response [CR]+partial response [PR]) in subjects with relapsed or refractory cHL.

### Secondary Objectives

Phase 1 and 2: To determine the safety profile of the compound of formula (I) in subjects with relapsed or refractory cHL; To determine the pharmacokinetics (PK) profile of the compound of formula (I) in subjects with relapsed or refractory cHL; To assess the effect of the compound of formula (I) on [¹⁸F]fluorodeoxyglucose(FDG)—positron emission tomography (PET) activity in subjects with relapsed or refractory cHL; To assess pharmacodynamic (PD) biomarkers of the compound of formula (I)in blood and tissue; To explore biomarkers predictive of response to the compound of formula (I); To explore PK/PD relationships with markers of pharmacological activity, efficacy, and treatment-emergent adverse events;

Phase 2: To determine the duration of response (DOR); To estimate progression-free survival (PFS).

### Study Design

This is an open-label, multicenter, dose-escalation, Phase ½ study to evaluate the clinical efficacy, safety and PK of the compound of formula (I). Up to 38 subjects are enrolled in the Phase 1 portion of the study and approximately 30 subjects or more are enrolled in the Phase 2 portion of the study. During the Phase 1 portion of the study, dose escalation of the compound of formula (I) starts at 150 mg (Cohort 1) once daily up to the maximum tolerated dose (MTD) or the highest planned dose (600 mg once daily); twice daily dosing may also be performed if deemed necessary. A Study Evaluation Team (SET) reviews all available data after 1 cycle of treatment for each cohort before any additional dose escalation occurs. The SET also determines the recommended Phase 2 dose.

There are 3 Periods for this study: a Screening Period (from signing of informed consent until immediately before dosing), an open-label Treatment Period (from the first dose until the End-of-Treatment Visit), and a Follow-up Period (after the End-of-Treatment Visit). All subjects participate in the Screening and Treatment Period. Only subjects who discontinue study drug before disease progression or discontinue due to treatment-related Grade 3 or higher toxicity continue in the Follow-up Period. Subjects are administered the compound of formula (I) continuously until disease progression, or unacceptable toxicity (based on investigator assessment). Subjects who achieve a CR may discontinue after an additional 2 cycles of treatment beyond the confirmed CR based on investigator decision. If a subject who achieved a CR and discontinued treatment is subsequently assessed with disease progression, the subject is allowed to re-initiate treatment at the previously administered the compound of formula (I) dose provided that conditions specified in the protocol are met. The end of the study is defined as the last follow-up visit of the last subject or termination of the study by the sponsor.

The subject population comprises men and women aged 18 years of age or older with a histopathologically confirmed initial diagnosis of cHL and who have disease that has relapsed or is refractory after at least 1 appropriate therapy.

### Dosage and Administration

The compound of formula (I) is self-administered orally once daily on a continuous dosing regimen preferably in the morning shortly after breakfast at approximately the same time each day. Each treatment cycle is 21 days. During the dose escalation phase of the study, the starting dose for the first cohort (Cohort 1) is 150 mg once a day. Twice daily dosing may also be performed if deemed necessary. Following the dose escalation phase, a decision is made by the SET regarding the dose for Phase 2 evaluation.

### Efficacy Evaluations

Disease response is assessed according to the Revised Response Criteria for Malignant Lymphoma. The analysis of response rate includes data from physical examination, computed tomography (CT) and FDG-PET or magnetic resonance imaging (MRI) scan, if applicable. The evaluations are performed throughout the study for each subject using the same method of assessment.

### Safety Evaluations

Safety assessments include vital signs, general physical examination, adverse events, concomitant medication review, electrocardiograms (ECGs), pregnancy testing, and laboratory testing. Adverse events (including laboratory abnormalities reported as adverse events) that occur between the signing of the informed consent form through 30 days following the last dose of the compound of formula (I) are collected. The intensity (severity) of adverse events are assessed using National Cancer Institute Common Terminology Criteria for Adverse Events (NCI CTCAE) version 4.03.

### Statistical Methods

The sample size estimated for the Phase 1 portion of the study is based on the utilization of a traditional 3+3 design and an additional 8 subjects at the MTD dose. The sample size estimated for Phase 2 assumes a 30% overall response rate. A sample size of 27 response-evaluable subjects at specified dose level (including subjects treated at the same dose during Phase 1 and in the expansion cohort if deemed appropriate) to provide a 2-sided 95% confidence interval (CI) (14%; 50%). To account for a dropout rate of approximately 10%, up to 30 subjects treated in total at the recommended Phase 2 dose (including subjects treated at the same dose during Phase 1 and in the expansion cohort) are enrolled.

The primary efficacy endpoint in Phase 2 is the overall response rate, defined as the proportion of response-evaluable subjects who achieve CR or PR. An estimate of the overall response rate is presented with an exact 2-sided 95% CI. Secondary endpoints include PFS and DOR. Progression-free survival and DOR evaluated using the Kaplan-Meier method. The estimated median PFS and median DOR along with the corresponding 95% CIs are presented. An exploratory efficacy analysis is performed by pooling data from Phase 1 and 2 subjects receiving the same dose of study drug. All safety analyses are performed on the Treated Population, which include all subjects who receive at least 1 dose of study drug. Serious adverse events are summarized separately. The reporting of safety data includes the incidence and type of adverse events. Clinical laboratory results and the proportion of subjects with clinically important changes from baseline values in vital signs are summarized.

While the foregoing specification teaches the principles of the present invention, with examples provided for the purpose of illustration, it is understood that the practice of the invention encompasses all of the usual variations, adaptations and/or modifications as come within the scope of the following claims and their equivalents.

We claim:

1. A method for the treatment of Hodgkin's Lymphoma comprising administering to a patient in need thereof, a therapeutically effective amount of a compound of formula (I) 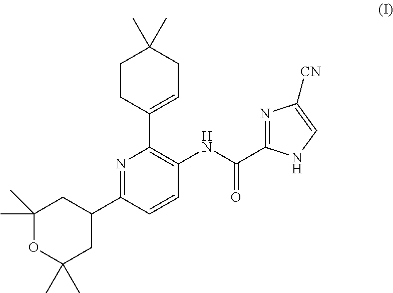 [SMILES: CC1(C)CC=C(c2nc(C3CC(C)(C)OC(C)(C)C3)ccc2NC(=O)c2nc(C#N)c[nH]2)CC1]
or a tautomer or pharmaceutically acceptable salt thereof.

2. The method according to claim 1, wherein the compound of formula (I) or a tautomer or pharmaceutically acceptable salt thereof is administered in a daily dosage amount in the range of from about 0.1 mg/kg to about 15.0 mg/kg.

3. The method according to claim 2, wherein the compound of formula (I) or a tautomer or pharmaceutically acceptable salt thereof is administered in a daily dosage amount in the range of from about 1.0 mg/kg to about 10.0 mg/kg.

4. The method according to claim 1, wherein the compound of formula (I) i or a tautomer or pharmaceutically acceptable salt thereof s administered in a daily dosage amount in the range of from about 10 mg to about 1000 mg.

5. The method according to claim 4, wherein the compound of formula (I) or a tautomer or pharmaceutically acceptable salt thereof is administer in a daily dosage amount in the range of from about 100 mg to about 600 mg.

6. The method according to claim 1, wherein the compound of formula (I) or a tautomer or pharmaceutically acceptable salt thereof is administered orally or intravenously.

7. A method for the treatment of Hodgkin's Lymphoma comprising administering to a patient in need thereof, a therapeutically effective amount of combination therapy comprising
(a) a compound of formula (I) 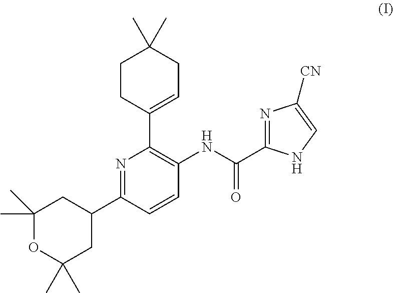 [SMILES: CC1(C)CC=C(c2nc(C3CC(C)(C)OC(C)(C)C3)ccc2NC(=O)c2nc(C#N)c[nH]2)CC1]
or a tautomer or pharmaceutically acceptable salt thereof; and
(b) a chemotherapy regimen.

8. The method according to claim 7, wherein the chemotherapy regimen is selected from the group consisting of ABVD, BEACOPP and Stanford V.

9. A method for the treatment of Hodgkin's Lymphoma comprising administering to a patient in need thereof, a therapeutically effective amount of combination therapy comprising
(a) a compound of formula (I) 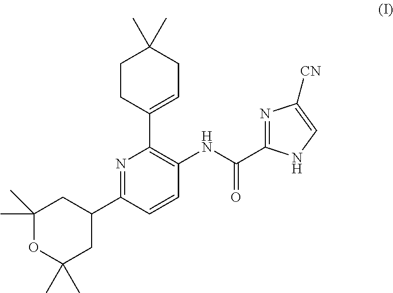 [SMILES: CC1(C)CC=C(c2nc(C3CC(C)(C)OC(C)(C)C3)ccc2NC(=O)c2nc(C#N)c[nH]2)CC1]
or a tautomer or pharmaceutically acceptable salt thereof; and
(b) one or more chemotherapeutic agent.

10. The method according to claim 9, wherein each chemotherapeutic agent is independently selected from the group consisting of doxorubicin, bleomycin, vinblastine, vincristine, dacarbazine, mechlorethamine, cyclophosphamide, procarbazine, etoposide or prednisone.

11. The method according to claim 9, wherein the combination therapy comprises the compound of formula (I) or a tautomer or pharmaceutically acceptable salt thereof; and doxorubicin.

12. The method according to claim 9, wherein the combination therapy comprises the compound of formula (I) or a tautomer or pharmaceutically acceptable salt thereof; doxorubicin, bleomycin and vinblastine.

13. A pharmaceutical composition for the treatment of Hodgkin's Lymphoma comprising
(a) a compound of formula (I) 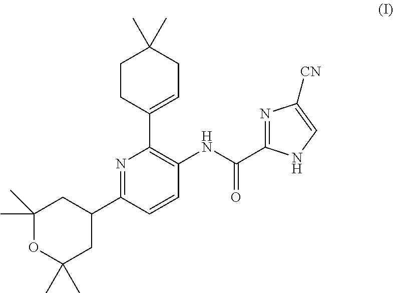 [SMILES: CC1(C)CC=C(c2nc(C3CC(C)(C)OC(C)(C)C3)ccc2NC(=O)c2nc(C#N)c[nH]2)CC1]
or a tautomer or pharmaceutically acceptable salt thereof;
(b) optionally one or more chemotherapeutic agent or chemotherapy regimen; and
(c) at least one pharmaceutically acceptable carrier.

14. (canceled)

15. (canceled)

16. (canceled)

17. (canceled)

18. (canceled)
